(19) United States
(12) Patent Application Publication
(10) Pub. No.: US 2019/0320899 A1
(43) Pub. Date: Oct. 24, 2019

(54) PROXIMITY BASED DATA ACQUISITION AND DISPLAY

(71) Applicant: Covidien LP, Mansfield, MA (US)

(72) Inventors: Amit Mukherjee, Elkridge, MD (US); Shashidhar Yamsani, Annapolis, MD (US); Ankita Mishra, Crofton, MD (US); Gokul Krishna Vangara, Hyderabad (IN)

(21) Appl. No.: 15/958,341

(22) Filed: Apr. 20, 2018

(51) Int. Cl. A61B 5/00; G16H 10/60; H04W 4/80; H04L 29/06

(52) U.S. Cl. CPC A61B 5/002; H04L 63/0876; H04W 4/80; G16H 10/60

(57) ABSTRACT

Methods, systems, and devices that support proximity based data acquisition and display are described. Methods may include determining a proximity between a patient identification tag and a user device to display patient-specific physiological data to authorized clinicians. The method may also include determining a proximity between an identification tag of a central monitoring station (CMS) and a user device to display physiological data associated with one or more patients to authorized users of the CMS. In addition the method may grant, or revoke, access to patient physiological data based clinician-specific credentials.

### BACKGROUND

The following relates generally to receiving physiological data associated with a patient at a user device, and more specifically to proximity based data acquisition and display.

In a healthcare facility such as a hospital, physiological parameters of the patient (e.g., heart rate, respiratory rate, blood pressure) may be monitored by one or more medical devices. The medical devices may be battery powered and may wirelessly transmit measured patient data over a wireless network within the hospital, thereby allowing the patient to move freely through the hospital while being monitored. Clinicians may remotely monitor the patient by accessing the patient data at a central nurse station or on any web enabled device connected to the network (e.g., smartphone or tablet).

In some cases, clinicians may access patient data (e.g., physiological data) being transmitted from patient monitoring devices. However, the patient data may not be filtered based on the credentials of the clinician receiving the data. In some cases, a variety of clinicians (e.g., doctors, nurses, technicians, etc.) may be able to receive the entirety of physiological data associated with a patient although only a subset of clinicians may be authorized to view the data. Additionally, viewing the entirety of a patient's data may be both time consuming and highly-complex based on the amount of data readily available. Therefore, improvements in patient data acquisition are desired.

### SUMMARY

The described features generally relate to methods, systems, devices, or apparatuses that support proximity based data acquisition and display. A user device may determine a proximity between the user device and an identification tag associated with the patient, the user device comprising authentication information of a user of the user device. Upon determining the proximity between the user device and the identification tag associated with the patient, the user device may authenticate the user device based at least in part on the proximity and an association between the identification tag and the authentication information. The user device may then transmit a signal to a sensor associated with the patient based at least in part on authenticating the user device. Upon transmitting the signal to the sensor associated with the patient, the user device may receive physiological data from the sensor associated with the patient based at least in part on transmitting the signal.

Methods and apparatuses are described that support proximity based data acquisition and display. A method may include receiving a subset of physiological data from the sensor associated with the patient based at least in part on a user type of the user of the user device. Additionally, the method may include de-authenticating the user device after receiving the physiological data based at least in part on the proximity between the user device and the identification tag exceeding a threshold. In some examples, the user type may comprise a doctor, a nurse, a physician assistant, or a technician. In other examples, authenticating the user device may be based at least in part on an angle of arrival of the user device. Additionally or alternatively, the identification tag may comprise a radio-frequency identification (RFID) tag.

A central monitoring station may receive physiological data associated with the patient. Upon receiving the physiological data associated with the patient, the central monitoring station may determine a proximity between a user device of a clinician and an identification tag of the central monitoring station. The central monitoring station may then authenticate the user device based at least in part on the proximity and an association between the identification tag and authentication information of the clinician. Upon authenticating the user device, the central monitoring station may display the physiological data associated with the patient based at least in part on authenticating the user device.

Methods and apparatuses are described that support proximity based data acquisition and display. A method may include granting access to a user profile associated with the clinician based at least in part on authenticating the user device, wherein the physiological data associated with the patient is displayed within the user profile. Additionally, the method may include revoking access to the user profile associated with the clinician based at least in part on the proximity exceeding a threshold. In some examples, the method may include displaying physiological data associated with a plurality of patients within the user profile, wherein the physiological data associated with the plurality of patients is displayed based at least in part on authenticating the user device.

In other examples, the method may include displaying a subset of the physiological data associated with the patient based at least in part on a credential of the clinician. Additionally or alternatively, the method may include transmitting a broadcast message to one or more sensors that indicates a configuration update for the one or more sensors. In some examples, the physiological data associated with the patient may be received based at least in part on the proximity between the user device and the identification tag of the central monitoring station. In other examples, authenticating the user device may be based at least in part on a media access control (MAC) address associated with the user device.

Certain examples of the present disclosure may include some, all, or none of the above advantages or features. One or more other technical advantages or features may be readily apparent to those skilled in the art from the figures, descriptions, and claims included herein. Moreover, while specific advantages or features have been enumerated above, various examples may include all, some, or none of the enumerated advantages or features.

Further scope of the applicability of the described methods and systems will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

FIG. 1 illustrates an example of a system for receiving physiological data associated with a patient at a user device that supports proximity based data acquisition and display in accordance with aspects of the present disclosure.

FIG. 2 illustrates an system that supports proximity based data acquisition and display in accordance with aspects of the present disclosure.

FIG. 3 illustrates a flow diagram that supports proximity based data acquisition and display in accordance with aspects of the present disclosure.

FIG. 4 illustrates a flow diagram that supports proximity based data acquisition and display in accordance with aspects of the present disclosure.

FIGS. 5 through 7 show block diagrams of a user device that supports proximity based data acquisition and display in accordance with aspects of the present disclosure.

FIG. 8 illustrates a block diagram of a system including a user device that supports proximity based data acquisition and display in accordance with aspects of the present disclosure.

FIGS. 9 through 11 show block diagrams of a central monitoring system that supports proximity based data acquisition and display in accordance with aspects of the present disclosure.

FIG. 12 illustrates a block diagram of a system including a central monitoring station that supports proximity based data acquisition and display in accordance with aspects of the present disclosure.

FIGS. 13 and 14 illustrate methods for proximity based data acquisition and display in accordance with aspects of the present disclosure.

### DETAILED DESCRIPTION

In a healthcare facility, a variety of monitoring devices may monitor physiological parameters of a patient such as heart rate, blood oxygen saturation levels, respiratory rate, glucose level, etc. The physiological data may be collected and may be provided to a variety of clinicians (e.g., doctors, nurses, technicians, etc.). However, the entirety of patient physiological data is commonly provided to any one clinician. Stated another way, a clinician—whether a doctor, nurse, or technician—may receive a same set of patient physiological data. Consequently, patient physiological data may either be viewed by unauthorized clinicians, or viewing the entirety of a patient's data may be both time consuming and highly-complex. Accordingly, a system capable of displaying patient physiological data by authorizing a user device of a clinician based on his or her credentials and his or her proximity to a patient may result in more secure access to patient data, as well as a more efficient analysis of the patient's health by a clinician.

In a first example, a medical device (e.g., a sensor) may receive physiological data associated with a patient. The physiological data may include, for example, the patient's heart rate, blood pressure, respiratory rate, glucose level, etc. Upon receiving the physiological data, a user device may determine a proximity between the device and an identification tag associated with the patient. Based on the proximity, and one or more credentials provided to the user device by the user, the user device may be authenticated. Stated another way, a user (e.g., a clinician) may provide a user device with his or her credentials. Based on the credentials, and whether the user is within a predetermined distance from the patient, the user device may be authenticated to receive physiological data associated with the patient. If authenticated, the user device may transmit a signal (e.g., an indication) to the medical device. The signal may, essentially, alert the medical device that the user device is authorized to receive patient data the device is collecting. After the signal has been transmitted, the user device may receive the patient physiological data from the monitoring device.

In an additional, a central monitoring station (CMS) may receive physiological data associated with a patient. As described above, the physiological data may be received from the patient by a medical device (e.g., a sensor). The physiological data may include the patient's heart rate, blood pressure, respiratory rate, glucose level, etc. Upon receiving the physiological data, the CMS may determine a proximity between the CMS and a user device associated with a clinician. Based on the proximity, and one or more credentials provided to the user device by the clinician, the user device may be authenticated. Stated another way, a user (e.g., a clinician) may provide a user device with his or her credentials. Based on the credentials, and whether the user is within a predetermined distance from the CMS, the user device may be authenticated. Upon authenticating the user device, the CMS may display the patient physiological data received from the monitoring device.

Aspects of the disclosure are initially described in the context of a wireless patient monitoring system. Aspects of the disclosure are further illustrated by and described with reference to apparatus diagrams, system diagrams, and flowcharts that relate to proximity based data acquisition and display.

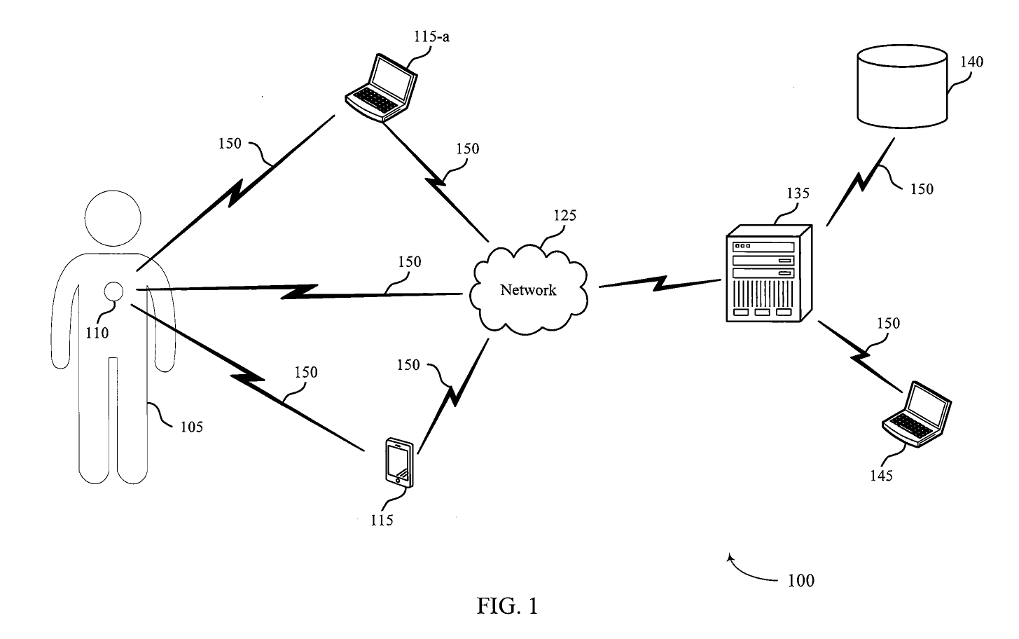

FIG. 1 illustrates an example of a wireless patient monitoring system 100 in accordance with various embodiments of the present disclosure. The wireless patient monitoring system 100 may include a patient 105 wearing, carrying, or otherwise coupled with a medical device 110. Although a single medical device 110 is shown, multiple medical devices 110 may be coupled to the patient 105. The patient 105 may be a patient in a hospital, nursing home, home care, a medical facility, or another care facility. The medical device 110 may transmit signals via wireless communications links 150 to computing devices 115 or to a network 125.

The medical device 110 may include one or more sensors configured to collect a variety of physiological parameters as well as information related to the location and movement of the patient 105. For example, the medical device 110 may include a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, a sensor which triangulates position from multiple local computing devices 115, or any other sensor configured to collect physiological, location, or motion data associated with the patient 105.

The medical device 110 may be coupled with the patient 105 in a variety of ways depending on the data being collected. For example, the medical device 110 may be directly coupled with the patient 105 (e.g., physically connected to the patient's chest, worn around the patient's wrist, attached to the patient's finger, or positioned over the patients nose or mouth). The data collected by the medical device 110 may be wirelessly transmitted to either the computing devices 115 or to the remote computing device 145 (via the network 125 and central station 135). Data transmission may occur via, for example, frequencies appropriate for a personal area network (such as Bluetooth, Bluetooth Low Energy (BLE), or IR communications) or local (e.g., wireless local area network (WLAN)) or wide area network (WAN) frequencies such as radio frequencies specified by IEEE standards (e.g., IEEE 802.15.4 standard, IEEE 802.11 standard (Wi-Fi), IEEE 802.16 standard (WiMAX), etc.).

Computing device 115 may be a wireless device such as a tablet, cellular phone, personal digital assistant (PDA), a dedicated receiver, or other similar device or a spatially distributed network of devices configured to receive signals from the medical device 110. Computing device 115-a may be a wireless laptop computer, a clinician Workstation on Wheels, or a smart hospital bed configured to receive signals from the medical device 110. The computing devices 115 may be in communication with a central station 135 via network 125.

The medical device 110 may also communicate directly with the central station 135 via the network 125. The central station 135 may be a server or a central nurse station located within the hospital or in a remote location. The central station 135 may be in further communication with one or more remote computing devices 145, thereby allowing a clinician to remotely monitor the patient 105. The central station 135 may also be in communication with various remote databases 140 where the collected patient data may be stored. In some cases, the remote databases 140 include electronic medical records (EMR) applications for storing and sharing patient data.

In some examples, a computing device 115-a may determine a distance (e.g., a proximity) between the computing device 115-a and a medical device 110 associated with the patient 105. The medical device 110 may be directly associated (e.g., worn) by the patient 105 and, in some examples, may be a radio frequency identification (RFID) tag. As described above, physiological data may include, for example, data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof associated with patient 105. In some examples, the computing device 115-a may include authentication information of a user of the computing device 115-a. For example, a user may provide credentials (e.g., login information) to gain access to the computing device 115-a. Such credentials may be an example of authentication information.

Upon determining a proximity between the computing device 115-a and the patient 105, the computing device 115-a may be authenticated. The authentication may occur based on the determined proximity and, in some examples, based on the authentication information provided to the computing device 115-a. For example, the computing device 115-a may be authenticated within a predefined radius (e.g., 5 feet) around the patient 105, and based in part on a credential provided by a user of the computing device 115-a. In some examples the authentication may occur at the computing device 115-a and, in other examples, the authentication may occur externally. For example, the authentication may occur at the central station 135 and an indication of the authentication may be provided to the computing device 115-a.

After being authenticated, the computing device 115-a may transmit a signal to a sensor associated with the patient 105. The sensor may be responsible for gathering physiological data associated with the patient through communication link 150. For example, a heart rate sensor associated with the patient 105 may receive the signal transmitted from the computing device 115-a. After transmitting the signal to the sensor 110, the computing device 115-a may receive sensor-specific physiological data. Stated another way, upon receiving a signal from the computing device 115-a, a sensor 110 associated with the patient 105 may transmit sensor-and-patient-specific physiological data to the computing device 115-a. Thus, the computing device 115-a may ultimately receive physiological data associated with a patient 105 based in part on authentication information (e.g., credentials) and its proximity to the patient 105.

In other examples, the central station 135 may receive physiological data associated with the patient 105. As described above, physiological data may include, for example, data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof associated with patient 105.

Upon receiving physiological data associated with the patient 105, the central station 135 may determine a distance (e.g., a proximity) between the central station 135 and a remote computing device 145. Based on the distance between the central station 135 and the remote computing device 145, the central station 135 may authenticate the remote computing device 145. In other examples, the remote computing device 145 may be authenticated based on authentication information (e.g., credentials) of a clinician operating the remote computing device 145. For example, the clinician may access (e.g., login to) the remote computing device 145, which may authenticate or aid in the authentication of the remote computing device 145. After the remote computing device 145 has been authenticated, the central station 135 may display the physiological data associated with the patient 105.

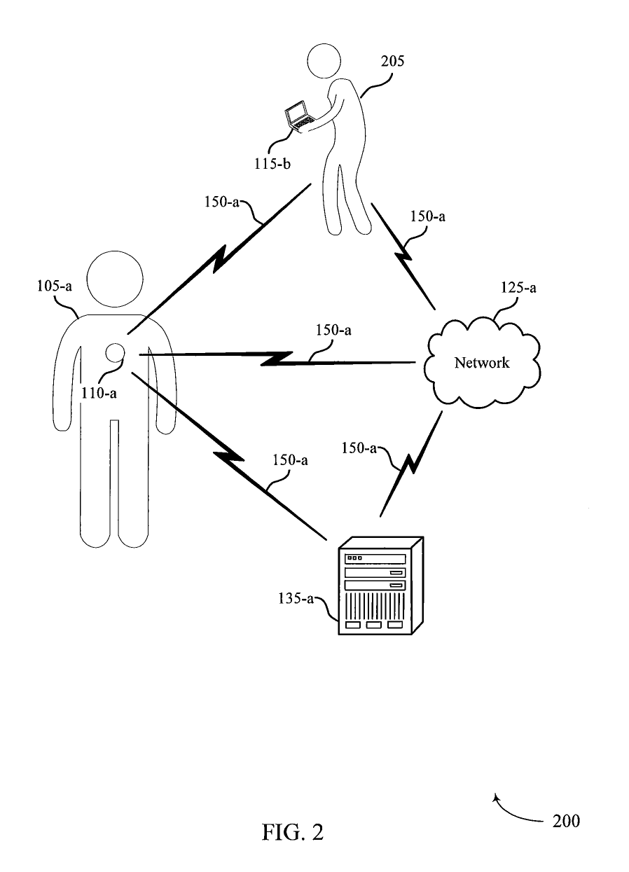

FIG. 2 illustrates an example of a patient monitoring system 200 that supports proximity based data acquisition and display. The patient monitoring system 200 may be an example of aspects of patient monitoring system 100 and may include a patient 105-a wearing, carrying, or otherwise coupled with a medical device 110-a. The medical device 110-a may be an example of medical device 110 as described with reference to FIG. 1, and may include one or more sensors configured to measure a variety of physiological parameters associated with the patient 105-a. Medical device 110-a may also be or may include identification information of the patient 105-a. In some examples, the patient monitoring system 200 may also include a clinician 205; a user device 115-b, which may be an example of computing device 115-a or 145 as described with reference to FIG. 1; a central monitoring station 135-a, which may be an example of central station 135 as described with reference to FIG. 1; and a network 125-a, which may be an example of network 125 as described with reference to FIG. 1. Each of the components illustrated may be connected via communication links 150-a, which may be examples of communication links 150 as described with reference to FIG. 1.

In some examples, medical device 110-a may receive physiological data associated with the patient 105-a. The physiological data may be received from medical device 110-a, which may be or may include one or more sensors to gather physiological data from patient 105-a. The data may be received, for example, continuously from the patient 105-a, or at one or more specific times. In some examples, the physiological data may include heart rate information of the patient 105-a. In other examples, the physiological data may include data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof associated with the patient 105-a. Additionally, as described above, the medical device 110-a may be directly coupled with the patient 105-a to receive the physiological data.

In some examples, medical device 110-a may include an identification tag associated with the patient. The identification tag may be patient-specific and therefore may be updated to include one or more identifying characteristics of a patient (e.g., his or her name). Thus medical device 110-a may be interchanged between patients, and the identification tag may be updated accordingly.

In some examples, the user device 115-b may include authentication information of a user of the device. For example, a user of user device 115-b may include a doctor, a nurse, a physician assistant, or a technician. Each user may possesses a credential that allows him or her to access patient-related information. For example, a credential of a doctor may allow him or her to access more detailed patient records than a credential possessed by a technician. The clinician-specific credentials may be received and/or authenticated by user device 115-b in a variety of ways. In some examples, user-specific credentials may be associated with a user profile of the user. Thus the user may enter login information—to access his or her user profile—to gain access to his or her credentials. In other examples, user authentication information may be presented to user device 115-b by fingerprint recognition or facial recognition. In other examples, temporary credentials may be assigned to a user, and may take the form of a temporary password or temporary login information entered into user device 115-b.

In some examples, the user device 115-b may determine a proximity between the user device 115-b and an identification tag associated with the patient 105-a. The proximity may be determined by a direct (e.g., wireless) communication between the user device 115-b and the identification tag. In other examples, the proximity may be determined via network connection 125-a.

Upon determining a proximity between the user device 115-b and the identification tag associated with the patient 105-a, the user device 115-b may be authenticated. Stated another way, if a user (e.g., clinician 205) of the user device 115-b possesses the requisite credentials, and the user device 115-b is within a predefined distance from the patient 105-a, the user device 115-b may be authenticated. For example, a user device 115-b may be required to be within 20 feet of a patient 105-a and the clinician 205 may be required to possess doctor-level credentials to view physiological data associated with patient 105-a. In some examples, to view physiological data associated with patient 105-a, the clinician 205 must be within 20 feet of patient 105-a and must possess doctor-level credentials. In other examples, to view physiological data associated with patient 105-a, the clinician 205 must be within 20 feet of patient 105- or must possess doctor-level credentials.

In other examples, the user device 115-b may be authenticated based in part on an angle of arrival of the user device 115-b. For example, clinician 205 may enter a patient's room and may proceed toward the patient. By approaching the patient 105-a (e.g., the angle of arrival of the user device 115-b), the user device 115-b may anticipate that the clinician 205 is intending to view physiological information associated with the particular patient 105-a. Thus the user device 115-b may be authenticated accordingly. In other examples, as described below, the user device 115-b may be de-authenticated. In some examples, the user device 115-b may be de-authenticated based on the angle of departure of the user device. For example, clinician 205 may begin to exit a patient's room (e.g., walk away from patient 105-a). The user device 115-b may anticipate that the clinician 205 is exiting the patient's room and may de-authenticate the user device 115-b accordingly.

After authenticating the user device—based in part on the proximity between the user device 115-b and the patient 105-a, the credentials of clinician 205, or both—the user device 115-b may transmit a signal to medical device 110-a. The signal may be transmitted directly to medical device 110-a via communication link 150-a or, in other examples, may be transmitted via communication link 150-a by way of network 125-a. The signal may serve as an indication to the medical device 110-a that the user device 115-b has been authenticated. Stated another way, the signal may indicate to the medical device 110-a that the user device 115-b may receive physiological information associated with the patient 105-a.

Upon transmitting the signal to the medical device 110-a, the user device 115-b may receive physiological data from the medical device 110-a. The physiological data may be received directly from the medical device 110-a via communication link 150-a or, in other examples, may be received via communication link 150-a by way of network 125-a. The user device 115-b may receive the physiological data and, in some examples, may display the data instantaneously (e.g., display the heart rate of patient 105-a).

As described above, a user device 115-b may be authenticated based in part on the credentials associated with a user (e.g., clinician 205). In some examples, the user's credentials may be such that he or she may view a subset of the physiological data associated with the patient 105-a. For example, a doctor may possess the highest form of credentials and may be able to view historical (e.g., longstanding) physiological data associated with the patient 105-a. Similarly, a nurse may possess a relatively high form of credentials as compared with a doctor, but may only be able to view physiological data associated with the patient 105-a from the time he or she came on shift (e.g., a subset of the physiological data). In other examples, a technician may possess the lowest form of credentials, but may still be able to view physiological data associated with the patient 105-a—in the form of instantaneous physiological data. Thus, in some examples, the credentials associated with clinician 205 may determine what, if any, patient physiological data may be viewed.

In some examples, the user device 115-b may receive the physiological data associated with the patient 105-a at a first time based on the user device 115-b being authenticated. The user device 115-b may receive the physiological data continuously so long as the user device 115-b remains authenticated. In other examples, the user device 115-b may attempt to receive the physiological data associated with the patient at a second time different from the first time. Stated another way, the medical device 110-a may attempt to transmit the physiological data to the user device 115-b at a second time different than the first time. The user device 115-b may successfully receive the data so long as the user device remains authenticated—e.g., it is within a predetermined proximity, possesses the requisite credentials, or both. If the user device 115-b remains authenticated, it may receive the physiological data. But, in some examples, the user device 115-b may have traveled out of range or may be in use by a clinician without requisite credentials. In such an example, the user device 115-b may not receive the physiological data or, in some examples, may prompt a user to re-enter his or her credentials to receive the data.

Additionally or alternatively, for example, the medical device 110-a may receive additional (e.g., different) physiological data associated with the patient 105-a. In some examples, the user device 115-b may be re-authenticated based on receiving the additional physiological data to ensure that a user of the user device 115-b possesses the requisite credentials. In other examples, if the user device 115-b is currently authenticated, the additional physiological information may be instantaneously transmitted to and received by the user device 115-b.

In other examples, the user device 115-b may be de-authenticated based on the occurrence of one or more events. As described above, a user (e.g., clinician 205) may cease using user device 115-b. For example, clinician 205 may logout of his or her user profile. This act may de-authenticate user device 115-b, as the device no longer possesses a user with the requisite credentials to view the patient's physiological data. In other examples, the user device 115-b may be de-authenticated based in part on the proximity between the user device 115-b and the identification tag associated with the patient exceeding a threshold distance. As described above, in some examples, user device 115-b may be authenticated if the proximity to the identification tag is less than 20 feet. For example, doctor may enter a patient's room (e.g., within 20 feet of the identification tag), view the patient's physiological data, and leave the room (e.g., and enter a different patient's room). In this example, the user device 115-b may be de-authenticated based on the clinician exiting the patient's room (e.g., exceeding 20 feet of the identification tag). In some examples, the user device 115-b may be re-authenticated as to a different patient based on the doctor entering the different patient's room (e.g., within 20 feet of a second identification tag).

In some examples, central monitoring station 135-a may receive physiological data associated with the patient 105-a. The physiological data may be received from medical device 110-a, which may be or may include one or more sensors to gather physiological data from patient 105-a. The data may be received, for example, continuously from the patient 105-a, or at one or more specific times. In some examples, the physiological data may include heart rate information of the patient 105-a. In other examples, the physiological data may include data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof associated with the patient 105-a. Additionally, as described above, the medical device 110-a may be directly coupled with the patient 105-a to receive the physiological data.

In some examples, central monitoring station 135-a may include an identification tag associated. The identification tag may be user-specific and therefore may reflect a current user or occupant of central monitoring station 135-a. Thus multiple users (e.g., clinicians) may use or occupy central monitoring station 135-a, and the identification tag may be updated accordingly.

As described above, the user device 115-b may include authentication information of a user of the device. For example, a user of user device 115-b may include a doctor, a nurse, a physician assistant, or a technician. Each user may possesses a credential that allows him or her to access patient-related information. For example, a credential of a doctor may allow him or her to access more detailed patient records than a credential possessed by a technician. The user-specific credentials may presented to user device 115-b in a variety of forms. In some examples, user-specific credentials may be associated with a user profile of the user. Thus the user may enter login information—to access his or her user profile—to gain access to his or her credentials. In other examples, the user-specific credentials may be presented to central monitoring station 135-a to access a user profile associate with the user (e.g., clinician 205). By accessing his or her user profile, clinician 205 may ultimately view physiological data associated with patient 105-a. In other examples, clinician 205 may view physiological data associated with a plurality of patients (e.g., patients on a particular floor or in a particular wing of a hospital). Ultimately, the physiological data may be displayed based in part on the authentication of the user device 115-b. As described above, user authentication information may be presented to central monitoring station 135-a by fingerprint recognition or facial recognition. In other examples, temporary credentials may be assigned to a user, and may take the form of a temporary password or temporary login information entered into central monitoring station 135-a.

In some examples, the central monitoring station 135-a may determine a proximity between the central monitoring station 135-a and a user device 115-b. The proximity may be determined by a direct (e.g., wireless) communication between the central monitoring station 135-a and the user device 115-b. In other examples, the proximity may be determined via network connection 125-a.

Upon determining a proximity between the central monitoring station 135-a and the user device 115-b, the user device 115-b may be authenticated. Stated another way, if a user (e.g., clinician 205) of the user device 115-b possesses the requisite credentials, and the user device 115-b is within a predefined distance from the central monitoring station 135-a, the user device 115-b may be authenticated. For example, a user device 115-b may be required to be within 20 feet of the central monitoring station 135-a and the clinician 205 may be required to possess doctor-level credentials to view physiological data associated with patient 105-a. In some examples, to view physiological data associated with patient 105-a, the user device 115-b must be within 20 feet of the central monitoring station 135-a and clinician 205 must possess doctor-level credentials. In other examples, to view physiological data associated with patient 105-a, the user device 115-b must be within 20 feet of central monitoring station 135-a or the clinician 205 must possess doctor-level credentials. Additionally or alternatively, the user device 115-b may be authenticated based at least in part on a media access control (MAC) address associated with the user device 115-b.

In other examples, the user device 115-b may be authenticated based in part on an angle of arrival of the user device 115-b. For example, a clinician 205 operating user device 115-b may approach the central monitoring station 135-a. By approaching the central monitoring station 135-a (e.g., the angle of arrival of the user device 115-b), the user device 115-b may anticipate that the clinician 205 is intending to view physiological information associated with the particular patient at central monitoring station 135-a. Thus the user device 115-b may be authenticated accordingly. In other examples, as described below, the user device 115-b may be de-authenticated. In some examples, the user device 115-b may be de-authenticated based on the angle of departure of the user device. For example, clinician 205 may begin to exit a central monitoring station 135-a (e.g., walk away from central monitoring station 135-a). The user device 115-b may anticipate that the clinician 205 is departing the central monitoring station 135-a and may de-authenticate the user device 115-b accordingly.

After authenticating the user device 115-b—based in part on the proximity between the user device 115-b and the central monitoring station 135-a, the credentials of clinician 205, or both—the central monitoring station 135-a may display physiological data associated with the patient (e.g., from the medical device 110-a). The physiological data may be received directly from the medical device 110-a via communication link 150-a or, in other examples, may be received via communication link 150-a by way of network 125-a. The central monitoring station 135-a may receive the physiological data and, in some examples, may display the data instantaneously (e.g., display the heart rate of patient 105-a).

In some examples, the central monitoring station 135-a may display the physiological data associated with the patient 105-a at a first time based on the user device 115-b being authenticated. The central monitoring station 135-a may display the physiological data continuously so long as the central monitoring station 135-a remains authenticated. In other examples, the central monitoring station 135-a may attempt to display the physiological data associated with the patient 105-a at a second time different from the first time. Stated another way, the medical device 110-a may attempt to transmit the physiological data to the central monitoring station 135-a at a second time different than the first time. The central monitoring station 135-a may successfully display the data so long as the central monitoring station 135-a remains authenticated—e.g., it is within a predetermined proximity of user device 115-b, the user device 115-b possesses the requisite credentials, or both. If the user device 115-b remains authenticated, the central monitoring station 135-a may display the physiological data. But, in some examples, the user device 115-b may have traveled out of range or may be in use by a clinician 205 without requisite credentials. In such an example, the central monitoring station 135-a may not display the physiological data or, in some examples, the user device 115-b may prompt a user to re-enter his or her credentials for the central monitoring station 135-a to display the physiological data.

Additionally or alternatively, for example, the medical device 110-a may receive additional (e.g., different) physiological data associated with the patient 105-a. In some examples, the user device 115-b may be re-authenticated based on receiving the additional physiological data to ensure that a user of the user device 115-b possesses the requisite credentials. In other examples, if the user device 115-b is currently authenticated, the additional physiological information may be instantaneously displayed by the central monitoring station 135-a.

In other examples, access to a user profile associated with clinician 205 may be revoked based on the occurrence of one or more events. As described above, a user (e.g., clinician 205) may cease using user device 115-b. For example, clinician 205 may logout of his or her user profile. This act may de-authenticate user device 115-b, as the device no longer possesses a user with the requisite credentials to view the patient's physiological data. In other examples, the user device 115-b may be de-authenticated based in part on the proximity between the user device 115-b and the identification tag associated with the central monitoring station 135-a exceeding a threshold distance. As described above, in some examples, user device 115-b may be authenticated if the proximity to the identification tag is less than 20 feet. For example, doctor may approach a central monitoring station 135-a (e.g., within 20 feet of the identification tag), view a patient's physiological data, and leave the central monitoring station 135-a. In this example, access to a user profile associated with the clinician 205 may be revoked based on the clinician exiting the central monitoring station 135-a (e.g., exceeding 20 feet of the identification tag). In some examples, the user device 115-b may be re-authenticated based on the doctor revisiting the central monitoring station 135-a.

Additionally or alternatively, the central monitoring station 135-a may transmit (e.g., via communication link 150-a) a broadcast message to one or more medical devices (e.g., sensors) 110-a. The broadcast message may indicate a configuration update for one or more medical devices 110-a. Stated another way, the broadcast message may synchronize one or more medical devices 110-a with the system 200. For example, an additional medical device 110-a may be added to the system 200. The broadcast message may transmit, to the additional medical device 110-a, a software update, timing parameters, network parameters, or the like to synchronize the additional medical device 110-a with the system 200. Accordingly, the additional medical device 110-a may operate in accordance with the methods described herein.

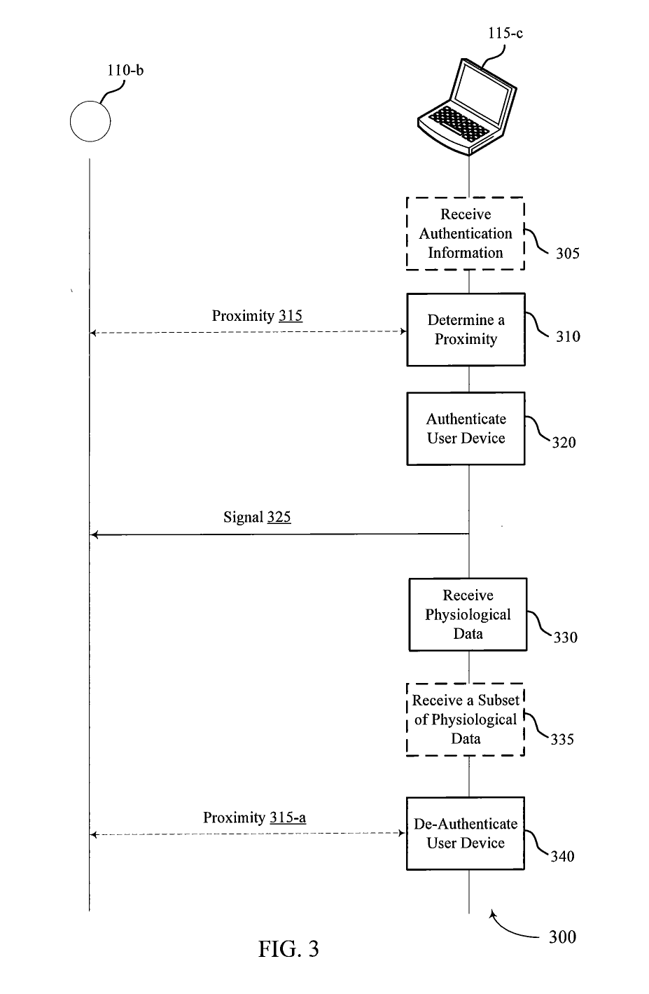

FIG. 3 illustrates an example process flow 300 that supports proximity based data acquisition and display. Process flow 300 may include medical device 110-b and user device 115-c, which may be respective examples of a medical device 110-a and user device 115-b as described in reference to FIG. 2. Alternative examples of the following may be implemented, where some steps are performed in a different order or not at all. Some steps may additionally include additional features not mentioned above.

Medical device 110-b may monitor a patient (e.g., patient 105-a as described with reference to FIG. 2) to receive physiological data associated with the patient. As described above, physiological data may include heart rate information of the patient or, in other examples, may include data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof. The physiological data received from medical device 110-b may ultimately be received my user device 115-c.

At block 305, user device 115-c may receive authentication information associated with a user (e.g., a clinician) of the user device 115-c. As described above, authentication information may identify the user as a doctor, a nurse, a physician assistant, or a technician. Specifically, the authentication information may be or may possess a credential that allows (or prohibits) the user to access patient-related information. For example, a credential of a doctor may allow him or her to access more detailed patient records than a credential possessed by a technician.

At block 310, user device 115-b may determine a proximity between the user device 115-c and the medical device 110-b. The proximity between the user device 115-b and the medical device 110-b may be a physical distance represented by proximity 315. Specifically, the user device 115-b may determine a proximity between an identification tag associated with the patient. As described above, the identification tag may be or may be associated with medical device 110-b. In some examples, the proximity between the user device 115-b may be determined by a direct (e.g., wireless) connection between the user device 115-b and the identification tag. In other examples, the determination may be made using a wireless network (e.g., network 125-a as described with reference to FIG. 2).

At block 320, the user device 115-c may be authenticated. As described above, the user device 115-c may be authenticated based in part on the proximity between the user device 115-c and the identification tag of the user (e.g., at block 310) and an association between the identification tag and the authentication information. Stated another way, a clinician may be assigned a particular patient and may possess the credentials to view his or her physiological data. Accordingly, the clinician may enter his or her authentication information into user device 115-c and, if the user device 115-c falls within an allowable proximity to the patient, the user device 115-c may be authenticated. Thus the user of the user device 115-c may ultimately be able to view the patient's physiological data.

Upon authenticating the user device 115-c, the user device 115-c may transmit a signal 325 to a sensor associated with the patient (e.g., to medical device 110-b). The signal may be or may include an indication that the user device 115-c has been authenticated. Accordingly, the signal may notify the medical device 110-b that the user device 115-c may receive physiological information associated with the patient.

At block 330, the user device 115-c may receive physiological data associated with the patient from medical device 110-b. The physiological data may be received directly from the medical device 110-b via a communication link (e.g., communication link 150-a as described with reference to FIG. 2) or, in other examples, may be received via a communication link by way of a network (e.g., network 125-a as described with reference to FIG. 2). The user device 115-c may receive the physiological data and, in some examples, may display the data instantaneously (e.g., display blood pressure data of the respective patient).

At block 335, the user device 115-c may receive a subset of physiological data from the medical device 110-b. In some examples, the user's credentials may be such that he or she may view a subset of the physiological data associated with the patient. Stated another way, the user of user device 115-c may not possess the requisite credentials to view the entirety of the physiological data associated with the patient. Thus, in some examples, the credentials associated with clinician may determine what, if any, patient physiological data may be viewed at the user device 115-c.

At block 340, the user device 115-c may be de-authenticated. For example, the user device 115-c may be de-authenticated based in part on the proximity between the user device 115-c and the identification tag associated with the patient exceeding a threshold distance represented by proximity 315-a. Thus if a user of user device 115-c walks away from a particular patient (e.g., exceeds a threshold distance), the user device 115-c may cease receiving physiological data associated with the patient.

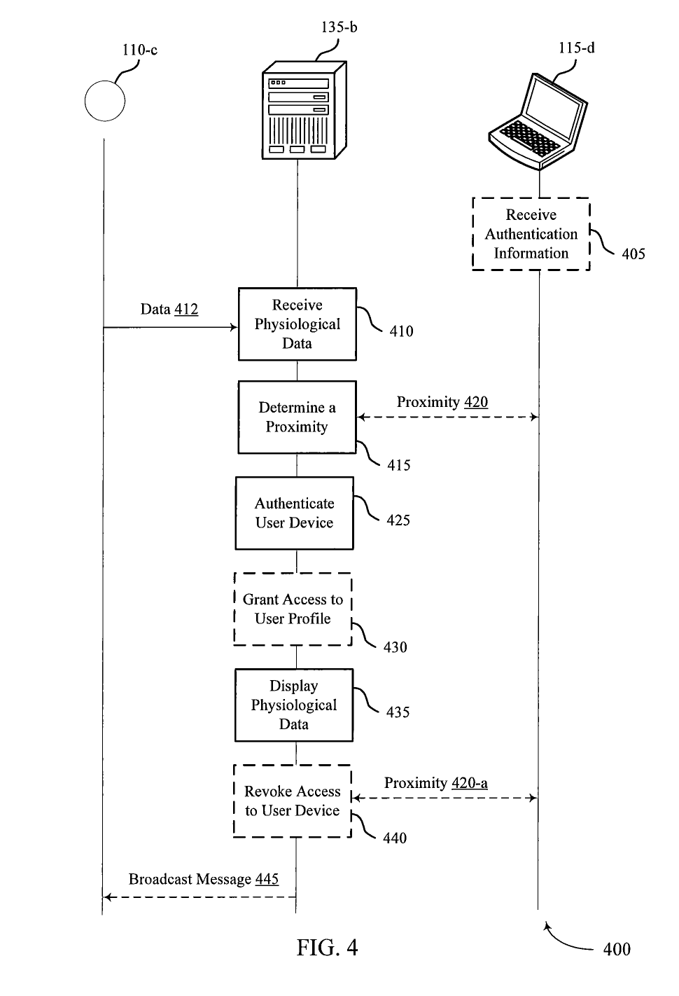

FIG. 4 illustrates an example process flow 400 that supports proximity based data acquisition and display. Process flow 400 may include medical device 110-c, central monitoring station 135-b, and user device 115-d, which may be respective examples of a medical device 110-a, central station 135-a, and user device 115-b as described in reference to FIG. 2. Alternative examples of the following may be implemented, where some steps are performed in a different order or not at all. Some steps may additionally include additional features not mentioned above.

Medical device 110-c may monitor a patient (e.g., patient 105-a as described with reference to FIG. 2) to receive physiological data associated with the patient. As described above, physiological data may include heart rate information of the patient or, in other examples, may include data received from a pulse oximetry (SpO2) sensor, a capnography sensor, a heart rate sensor, a blood pressure sensor, an electrocardiogram (ECG) sensor, a respiratory rate sensor, a glucose level sensor, a depth of consciousness sensor, a body temperature sensor, an accelerometer, a global positioning sensor, or a combination thereof. The physiological data received from medical device 110-b may ultimately be displayed by central monitoring station 135-b.

At block 405, user device 115-d may receive authentication information associated with a user (e.g., a clinician) of the user device 115-d. As described above, authentication information may identify the user as a doctor, a nurse, a physician assistant, or a technician. Specifically, the authentication information may be or may possess a credential that allows (or prohibits) the user to access patient-related information. For example, a credential of a doctor may allow him or her to access more detailed patient records than a credential possessed by a technician.

At block 410, central monitoring station 135-b may receive physiological data from medical device 110-c. The data may be represented by data 412. In some examples, the data may be received at central monitoring station 135-b from one or sensors associated with a patient. As described above, the physiological data may be or may include heart rate information, blood pressure information, the patient's repertory rate, glucose level, etc.

At block 415 central monitoring station 135-b may determine a proximity between the central monitoring station 135-b and the user device 115-d. The proximity between the central monitoring station 135-b and the user device 115-d may be a physical distance represented by proximity 420. Specifically, the central monitoring station 135-b may determine a proximity between an identification tag associated with central monitoring station 135-b and the user device 115-d. In some examples, the proximity between the central monitoring station 135-b and the user device 115-d may be determined by a direct (e.g., wireless) connection between the user device 115-b and the identification tag. In other examples, the determination may be made using a wireless network (e.g., network 125-a as described with reference to FIG. 2).

At block 425, the user device 115-d may be authenticated. As described above, the user device 115-d may be authenticated based in part on the proximity between the user device 115-d and the identification tag of the central monitoring station 135-b (e.g., at block 415) and an association between the identification tag and the authentication information. Stated another way, a clinician may be assigned a particular patient and may possess the credentials to view his or her physiological data. Accordingly, the clinician may enter his or her authentication information into user device 115-d and, if the user device 115-d falls within an allowable proximity to the central monitoring station 135-b, the user device 115-d may be authenticated. Thus the user of the user device 115-d may ultimately be able to view the patient's physiological data at the central monitoring station 135-b.

At block 430 a user of user device 115-d may be granted access to his or her user profile at central monitoring station 135-b. As described above, user-specific credentials may be associated with a user profile of the user. Thus the user may enter login information—to access his or her user profile—to gain access to his or her credentials. By accessing his or her user profile, a clinician may ultimately view physiological data associated with one or more patients (e.g., patients on a particular floor or in a particular wing of a hospital).

At block 435, the central monitoring station 135-b may display physiological data associated with the patient from medical device 110-d. The physiological data may be received directly from the medical device 110-c via a communication link (e.g., communication link 150-a as described with reference to FIG. 2) or, in other examples, may be received via a communication link by way of a network (e.g., network 125-a as described with reference to FIG. 2). The central monitoring station 135-b may display the physiological data and, in some examples, may display the data instantaneously (e.g., display blood pressure data of the respective patient).

At block 440, access of the user device 115-d may be revoked. For example, access of the user device 115-d may be revoked in part on the proximity between the user device 115-d and the identification tag associated with the central monitoring station 135-b exceeding a threshold distance represented by proximity 420-a. Thus if a user of user device 115-d walks away from the central monitoring station 135-b (e.g., exceeds a threshold distance), access of the user device 115-d may be revoked, resulting in the physiological information failing to be displayed at the central monitoring station 135-b.

Additionally or alternatively, the central monitoring station 135-a may transmit (e.g., via communication link 150-a) a broadcast message 445 to one or more medical devices (e.g., sensors) 110-c. As described above, the broadcast message may indicate a configuration update for one or more medical devices 110-c. Stated another way, the broadcast message 445 may synchronize one or more medical devices 110-c with the system 200. For example, an additional medical device 110-a may be added to the system (e.g., system 200 as described with reference to FIG. 2). The broadcast message 445 may transmit, to the additional medical device 110-c, a software update, timing parameters, network parameters, or the like to synchronize the additional medical device 110-c with the system. Accordingly, the additional medical device 110-c may operate in accordance with the methods described herein.

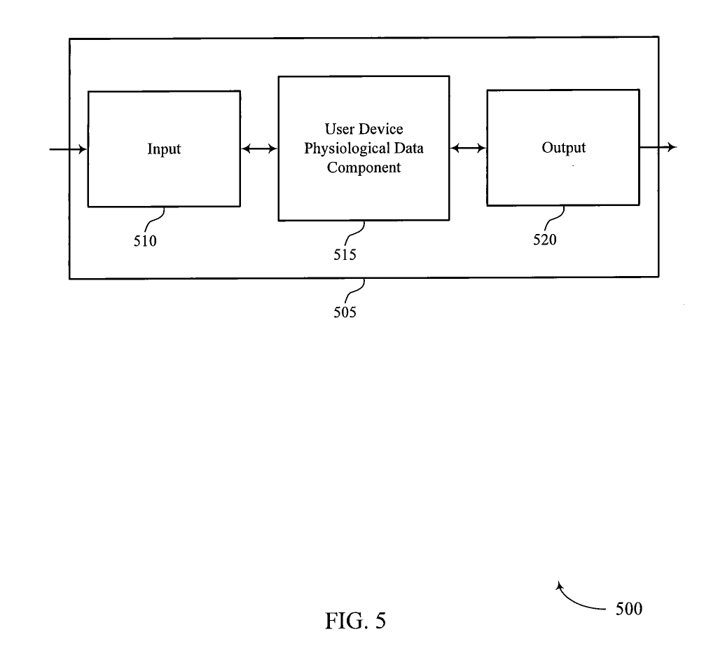

FIG. 5 shows a block diagram 500 of a device 505 that supports proximity based data acquisition and display in accordance with aspects of the present disclosure. Device 505 may be an example of aspects of a user device 115-b as described herein. Device 505 may include input 510, user device physiological data component 515, and output 520. Device 505 may also include a processor. Each of these components may be in communication with one another (e.g., via one or more buses).

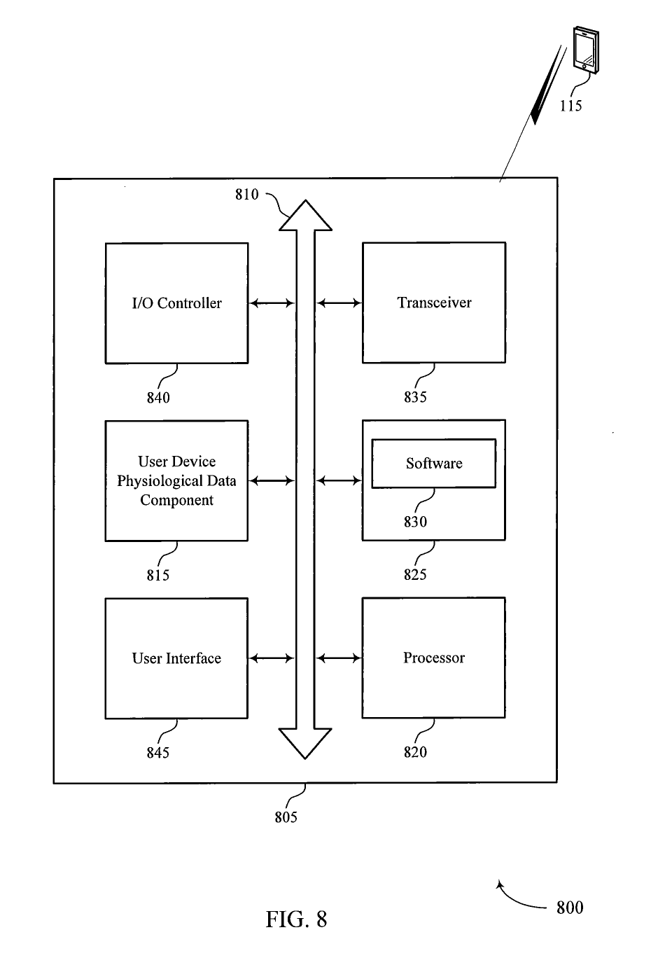

User device physiological data component 515 may be an example of aspects of the user device physiological data component 815 described with reference to FIG. 8.

User device physiological data component 515 and/or at least some of its various sub-components may be implemented in hardware, software executed by a processor, firmware, or any combination thereof. If implemented in software executed by a processor, the functions of the user device physiological data component 515 and/or at least some of its various sub-components may be executed by a general-purpose processor, a digital signal processor (DSP), an application-specific integrated circuit (ASIC), an field-programmable gate array (FPGA) or other programmable logic device, discrete gate or transistor logic, discrete hardware components, or any combination thereof designed to perform the functions described in the present disclosure. The user device physiological data component 515 and/or at least some of its various sub-components may be physically located at various positions, including being distributed such that portions of functions are implemented at different physical locations by one or more physical devices. In some examples, user device physiological data component 515 and/or at least some of its various sub-components may be a separate and distinct component in accordance with various aspects of the present disclosure. In other examples, user device physiological data component 515 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

User device physiological data component 515 may determine a proximity between the user device and an identification tag associated with the patient. In some examples, the user device may include authentication information of a user of the user device. The user device physiological data component 515 may authenticate the user device based on the proximity and an association between the identification tag and the authentication information. In some examples, the user device physiological data component 515 may transmit a signal to a sensor associated with the patient based on authenticating the user device. In other examples, the user device physiological data component 515 may receive physiological data from the sensor associated with the patient based on transmitting the signal.

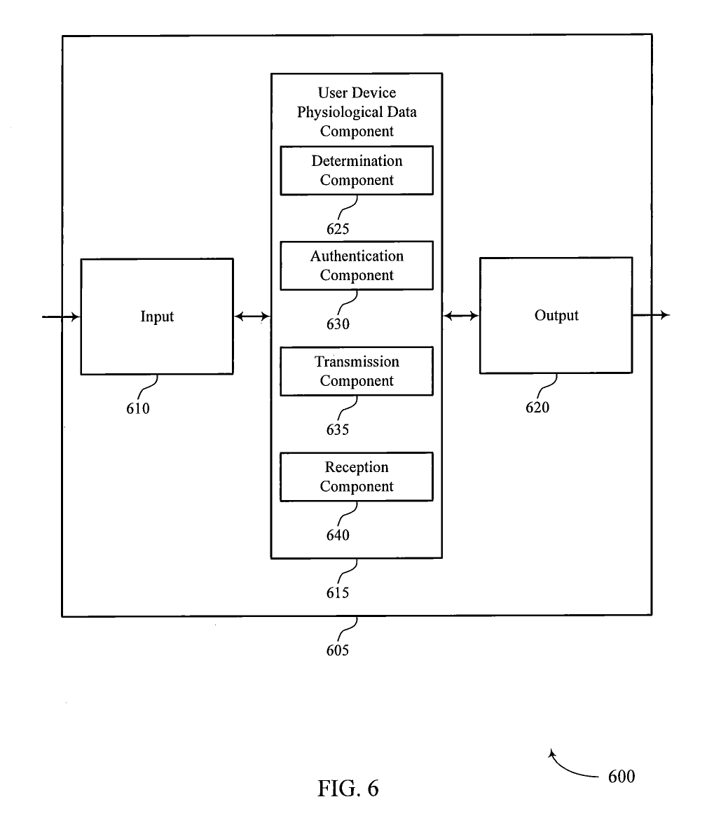

FIG. 6 shows a block diagram 600 of a device 605 that supports proximity based data acquisition and display in accordance with aspects of the present disclosure. Device 605 may be an example of aspects of a device 505 or a user device 115-b as described with reference to FIG. 5. Device 605 may include input 610, user device physiological data component 615, and output 620. Device 605 may also include a processor. Each of these components may be in communication with one another (e.g., via one or more buses).

User device physiological data component 615 may be an example of aspects of the user device physiological data component 815 described with reference to FIG. 8.

User device physiological data component 615 may include determination component 625, authentication component 630, transmission component 635, and reception component 640. Determination component 625 may determine a proximity between the user device and an identification tag associated with the patient. In some examples, the user device may include authentication information of a user of the user device.

Authentication component 630 may authenticate the user device based on the proximity and an association between the identification tag and the authentication information. In some examples, authentication component 630 may authenticate the user device based on an angle of arrival of the user device. In some cases, the identification tag may include a radio-frequency identification (RFID) tag.

Transmission component 635 may transmit a signal to a sensor associated with the patient based on authenticating the user device.

Reception component 640 may receive physiological data from the sensor associated with the patient based on transmitting the signal and receive a subset of physiological data from the sensor associated with the patient based on a user type of the user of the user device. In some cases, the user type includes a doctor, a nurse, a physician assistant, or a technician.

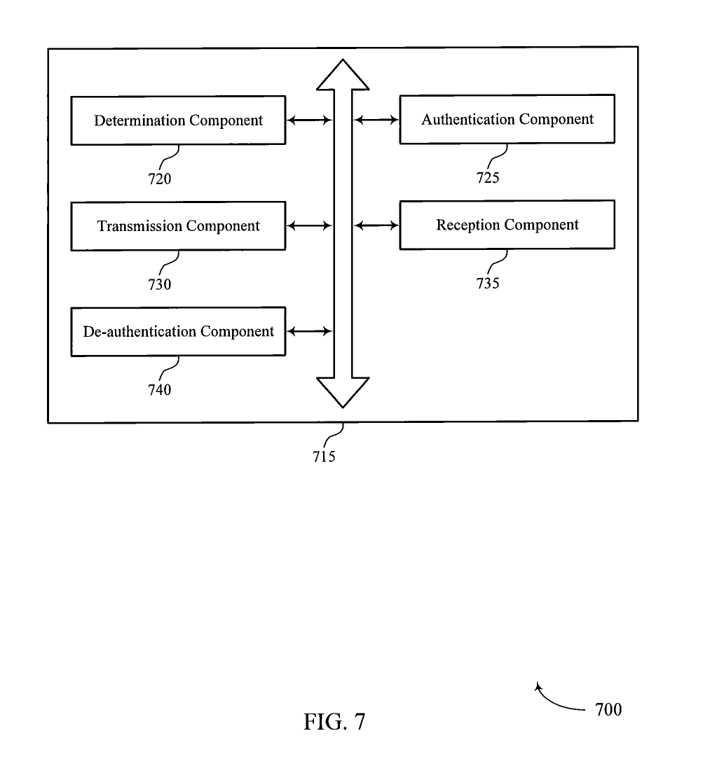

FIG. 7 shows a block diagram 700 of a user device physiological data component 715 that supports proximity based data acquisition and display in accordance with aspects of the present disclosure. The user device physiological data component 715 may be an example of aspects of a user device physiological data component 515, a user device physiological data component 615, or a user device physiological data component 815 described with reference to FIGS. 5, 6, and 8. The user device physiological data component 715 may include determination component 720, authentication component 725, transmission component 730, reception component 735, and de-authentication component 740. Each of these modules may communicate, directly or indirectly, with one another (e.g., via one or more buses).

Determination component 720 may determine a proximity between the user device and an identification tag associated with the patient. In some examples, the user device may include authentication information of a user of the user device.

Authentication component 725 may authenticate the user device based on the proximity and an association between the identification tag and the authentication information. In other examples, authentication component 725 may authenticate the user device based on an angle of arrival of the user device. In some cases, the identification tag includes a radio-frequency identification (RFID) tag.

Transmission component 730 may transmit a signal to a sensor associated with the patient based on authenticating the user device.

Reception component 735 may receive physiological data from the sensor associated with the patient based on transmitting the signal and receive a subset of physiological data from the sensor associated with the patient based on a user type of the user of the user device. In some cases, the user type includes a doctor, a nurse, a physician assistant, or a technician.

De-authentication component 740 may de-authenticate the user device after receiving the physiological data based on the proximity between the user device and the identification tag exceeding a threshold.

FIG. 8 shows a diagram of a system 800 including a device 805 that supports proximity based data acquisition and display in accordance with aspects of the present disclosure. Device 805 may be an example of or include the components of device 505, device 605, or a user device 115-b as described above, e.g., with reference to FIG. 2. Device 805 may include components for bi-directional voice and data communications including components for transmitting and receiving communications, including user device physiological data component 815, processor 820, memory 825, software 830, transceiver 835, I/O controller 840, and user interface 845. These components may be in electronic communication via one or more buses (e.g., bus 810).

Processor 820 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, processor 820 may be configured to operate a memory array using a memory controller. In other cases, a memory controller may be integrated into processor 820. Processor 820 may be configured to execute computer-readable instructions stored in a memory to perform various functions (e.g., functions or tasks supporting proximity based data acquisition and display).

Memory 825 may include random access memory (RAM) and read only memory (ROM). The memory 825 may store computer-readable, computer-executable software 830 including instructions that, when executed, cause the processor to perform various functions described herein. In some cases, the memory 825 may contain, among other things, a basic input/output system (BIOS) which may control basic hardware or software operation such as the interaction with peripheral components or devices.

Software 830 may include code to implement aspects of the present disclosure, including code to support proximity based data acquisition and display. Software 830 may be stored in a non-transitory computer-readable medium such as system memory or other memory. In some cases, the software 830 may not be directly executable by the processor but may cause a computer (e.g., when compiled and executed) to perform functions described herein.

Transceiver 835 may communicate bi-directionally, via one or more antennas, wired, or wireless links as described above. For example, the transceiver 835 may represent a wireless transceiver and may communicate bi-directionally with another wireless transceiver. The transceiver 835 may also include a modem to modulate the packets and provide the modulated packets to the antennas for transmission, and to demodulate packets received from the antennas.

I/O controller 840 may manage input and output signals for device 805. I/O controller 840 may also manage peripherals not integrated into device 805. In some cases, I/O controller 840 may represent a physical connection or port to an external peripheral. In some cases, I/O controller 840 may utilize an operating system such as iOS®, ANDROID®, MS-DOS®, MS-WINDOWS®, OS/2®, UNIX®, LINUX®, or another known operating system. In other cases, I/O controller 840 may represent or interact with a modem, a keyboard, a mouse, a touchscreen, or a similar device. In some cases, I/O controller 840 may be implemented as part of a processor. In some cases, a user may interact with device 805 via I/O controller 840 or via hardware components controlled by I/O controller 840.

User interface 845 may enable a user to interact with device 805. In some embodiments, the user interface module 845 may include an audio device, such as an external speaker system, an external display device such as a display screen, or an input device (e.g., remote control device interfaced with the user interface module 845 directly or through the I/O controller module).

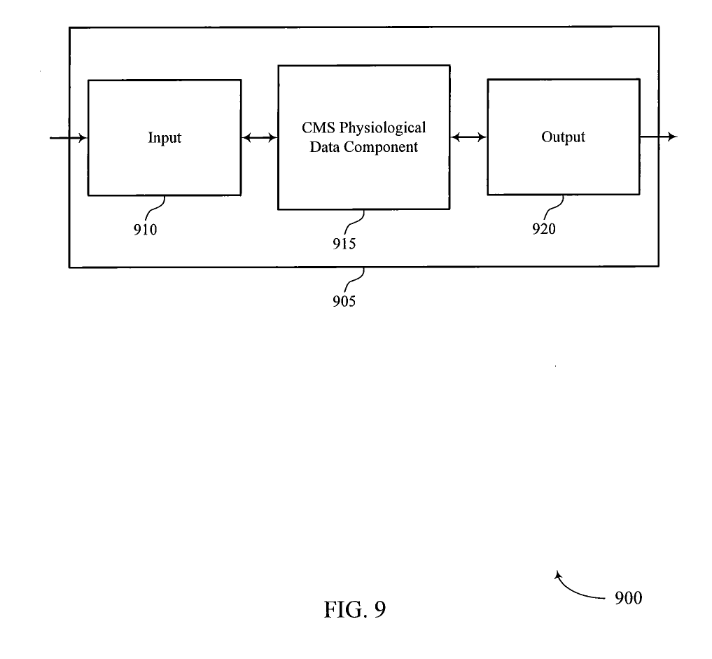

FIG. 9 shows a block diagram 900 of a device 905 that supports proximity based data acquisition and display in accordance with aspects of the present disclosure. Device 905 may be an example of aspects of a central monitoring station (CMS) 135-a as described herein. Device 905 may include input 910, CMS physiological data component 915, and output 920. Device 905 may also include a processor. Each of these components may be in communication with one another (e.g., via one or more buses).

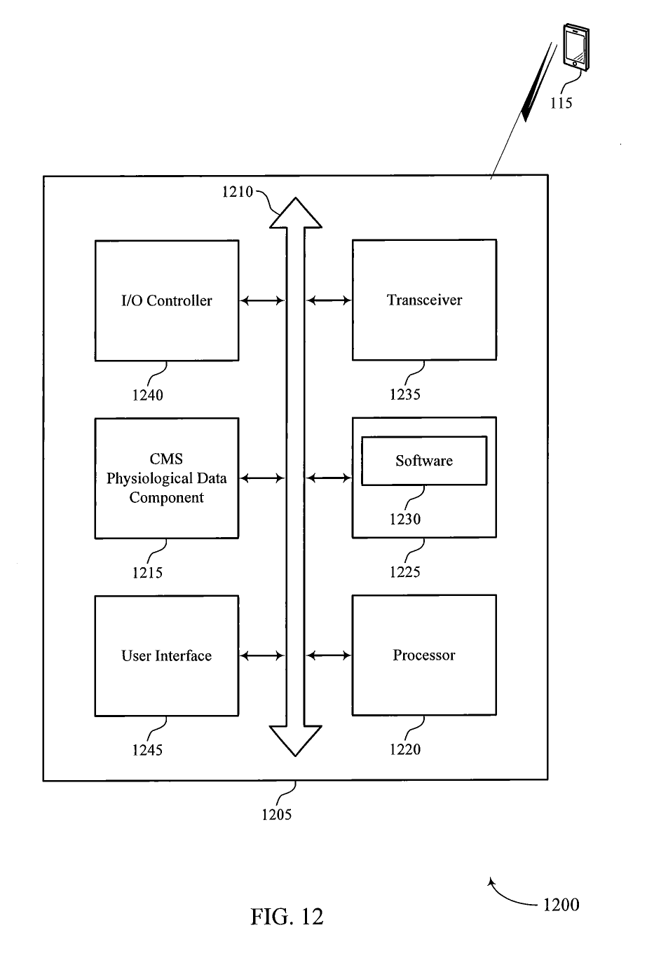

CMS physiological data component 915 may be an example of aspects of the CMS physiological data component 1215 described with reference to FIG. 12.

CMS physiological data component 915 and/or at least some of its various sub-components may be implemented in hardware, software executed by a processor, firmware, or any combination thereof. If implemented in software executed by a processor, the functions of the CMS physiological data component 915 and/or at least some of its various sub-components may be executed by a general-purpose processor, a DSP, an ASIC, an FPGA or other programmable logic device, discrete gate or transistor logic, discrete hardware components, or any combination thereof designed to perform the functions described in the present disclosure. The CMS physiological data component 915 and/or at least some of its various sub-components may be physically located at various positions, including being distributed such that portions of functions are implemented at different physical locations by one or more physical devices. In some examples, CMS physiological data component 915 and/or at least some of its various sub-components may be a separate and distinct component in accordance with various aspects of the present disclosure. In other examples, CMS physiological data component 915 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

CMS physiological data component 915 may receive physiological data associated with the patient, determine a proximity between a user device of a clinician and an identification tag of the central monitoring station, authenticate the user device based on the proximity and an association between the identification tag and authentication information of the clinician, and display the physiological data associated with the patient based on authenticating the user device.

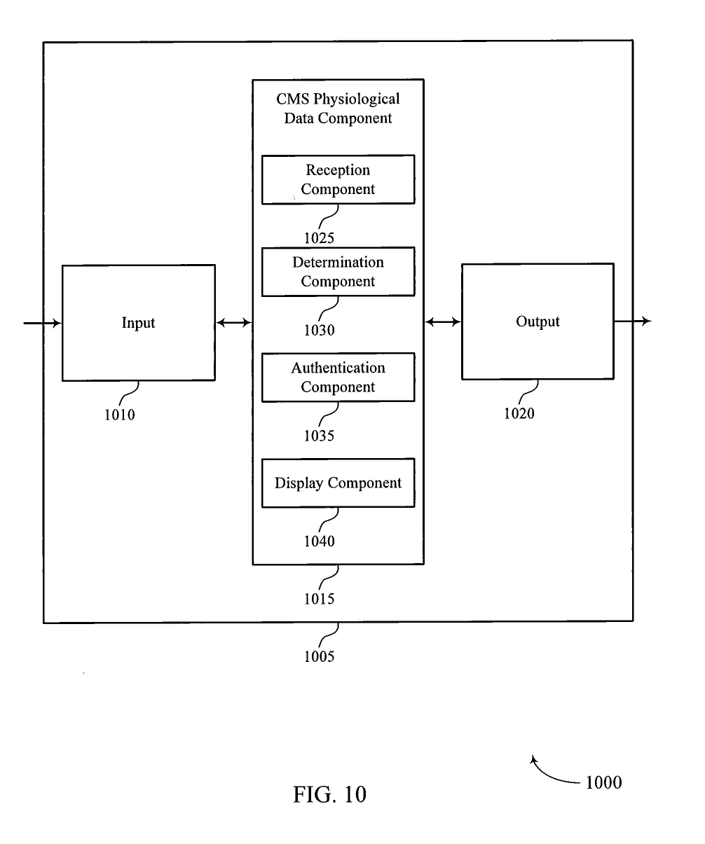

FIG. 10 shows a block diagram 1000 of a device 1005 that supports proximity based data acquisition and display in accordance with aspects of the present disclosure. Device 1005 may be an example of aspects of a device 905 or a central monitoring station 135-a as described with reference to FIG. 2. Device 1005 may include input 1010, CMS physiological data component 1015, and output 1020. Device 1005 may also include a processor. Each of these components may be in communication with one another (e.g., via one or more buses).

CMS physiological data component 1015 may be an example of aspects of the CMS physiological data component 1215 described with reference to FIG. 12.

CMS physiological data component 1015 may also include reception component 1025, determination component 1030, authentication component 1035, and display component 1040.

Reception component 1025 may receive physiological data associated with the patient. In some cases, the physiological data associated with the patient may be received based on the proximity between the user device and the identification tag of the CMS.

Determination component 1030 may determine a proximity between a user device of a clinician and an identification tag of the CMS.

Authentication component 1035 may authenticate the user device based on the proximity and an association between the identification tag and authentication information of the clinician. In some examples, authentication component 1035 may grant access to a user profile associated with the clinician based on authenticating the user device, where the physiological data associated with the patient is displayed within the user profile. In other examples, authentication component 1035 may authenticate the user device based on a media access control (MAC) address associated with the user device.

Display component 1040 may display the physiological data associated with the patient based on authenticating the user device. In some examples, display component 1040 may display physiological data associated with a set of patients within the user profile, where the physiological data associated with the set of patients is displayed based on authenticating the user device. In other examples, display component 1040 may display a subset of the physiological data associated with the patient based on a credential of the clinician.

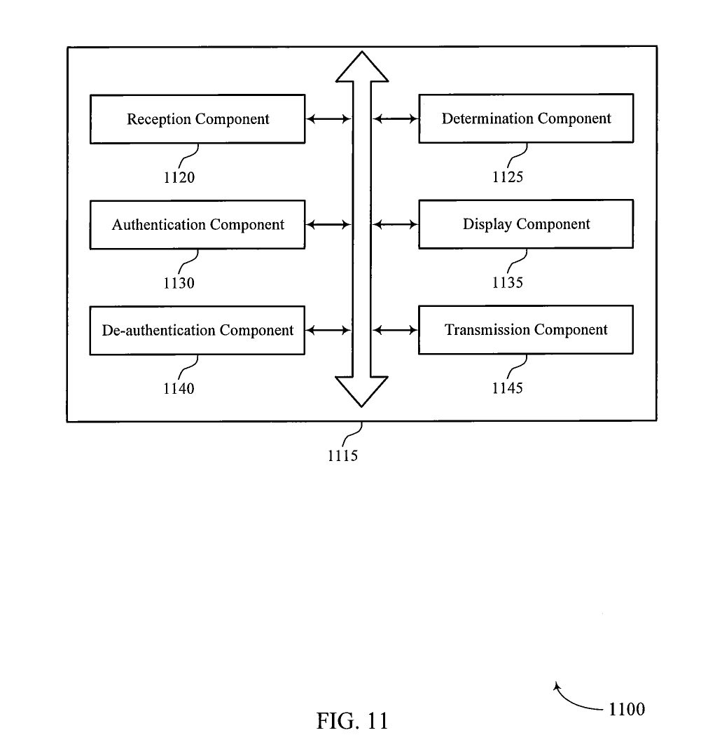

FIG. 11 shows a block diagram 1100 of a central monitoring station physiological data component 1115 that supports proximity based data acquisition and display in accordance with aspects of the present disclosure. The central monitoring station physiological data component 1115 may be an example of aspects of a central monitoring station physiological data component 1115 described with reference to FIGS. 9, 10, and 12. The central monitoring station physiological data component 1115 may include reception component 1120, determination component 1125, authentication component 1130, display component 1135, de-authentication component 1140, and transmission component 1145. Each of these modules may communicate, directly or indirectly, with one another (e.g., via one or more buses).

Reception component 1120 may receive physiological data associated with the patient. In some cases, the physiological data associated with the patient may be received based on the proximity between the user device and the identification tag of the central monitoring station.

Determination component 1125 may determine a proximity between a user device of a clinician and an identification tag of the central monitoring station.

Authentication component 1130 may authenticate the user device based on the proximity and an association between the identification tag and authentication information of the clinician. In other examples, authentication component 1130 may grant access to a user profile associated with the clinician based on authenticating the user device, where the physiological data associated with the patient is displayed within the user profile. In some examples, authentication component 1130 may authenticate the user device based on a MAC address associated with the user device.

Display component 1135 may display the physiological data associated with the patient based on authenticating the user device. In some examples, display component 1135 may display physiological data associated with a set of patients within the user profile, where the physiological data associated with the set of patients is displayed based on authenticating the user device. In other examples, display component 1135 may display a subset of the physiological data associated with the patient based on a credential of the clinician.

De-authentication component 1140 may revoke access to the user profile associated with the clinician based on the proximity exceeding a threshold.

Transmission component 1145 may transmit a broadcast message to one or more sensors that indicates a configuration update for the one or more sensors.

FIG. 12 shows a diagram of a system 1200 including a device 1205 that supports proximity based data acquisition and display in accordance with aspects of the present disclosure. Device 1205 may be an example of or include the components of central monitoring station 135-a as described above, e.g., with reference to FIG. 2. Device 1205 may include components for bi-directional voice and data communications including components for transmitting and receiving communications, including CMS physiological data component 1215, processor 1220, memory 1225, software 1230, transceiver 1235, I/O controller 1240, and user interface 1245. These components may be in electronic communication via one or more buses (e.g., bus 1210).

Processor 1220 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, processor 1220 may be configured to operate a memory array using a memory controller. In other cases, a memory controller may be integrated into processor 1220. Processor 1220 may be configured to execute computer-readable instructions stored in a memory to perform various functions (e.g., functions or tasks supporting proximity based data acquisition and display).

Memory 1225 may include RAM and ROM. The memory 1225 may store computer-readable, computer-executable software 1230 including instructions that, when executed, cause the processor to perform various functions described herein. In some cases, the memory 1225 may contain, among other things, a BIOS which may control basic hardware or software operation such as the interaction with peripheral components or devices.

Software 1230 may include code to implement aspects of the present disclosure, including code to support proximity based data acquisition and display. Software 1230 may be stored in a non-transitory computer-readable medium such as system memory or other memory. In some cases, the software 1230 may not be directly executable by the processor but may cause a computer (e.g., when compiled and executed) to perform functions described herein.

Transceiver 1235 may communicate bi-directionally, via one or more antennas, wired, or wireless links as described above. For example, the transceiver 1235 may represent a wireless transceiver and may communicate bi-directionally with another wireless transceiver. The transceiver 1235 may also include a modem to modulate the packets and provide the modulated packets to the antennas for transmission, and to demodulate packets received from the antennas.

I/O controller 1240 may manage input and output signals for device 1205. I/O controller 1240 may also manage peripherals not integrated into device 1205. In some cases, I/O controller 1240 may represent a physical connection or port to an external peripheral. In some cases, I/O controller 1240 may utilize an operating system such as iOS®, ANDROID®, MS-DOS®, MS-WINDOWS®, OS/2®, UNIX®, LINUX®, or another known operating system. In other cases, I/O controller 1240 may represent or interact with a modem, a keyboard, a mouse, a touchscreen, or a similar device. In some cases, I/O controller 1240 may be implemented as part of a processor. In some cases, a user may interact with device 1205 via I/O controller 1240 or via hardware components controlled by I/O controller 1240.

User interface 1245 may enable a user to interact with device 1205. In some embodiments, the user interface 1245 may include an audio device, such as an external speaker system, an external display device such as a display screen, or an input device (e.g., remote control device interfaced with the user interface 1245 directly or through the I/O controller module).

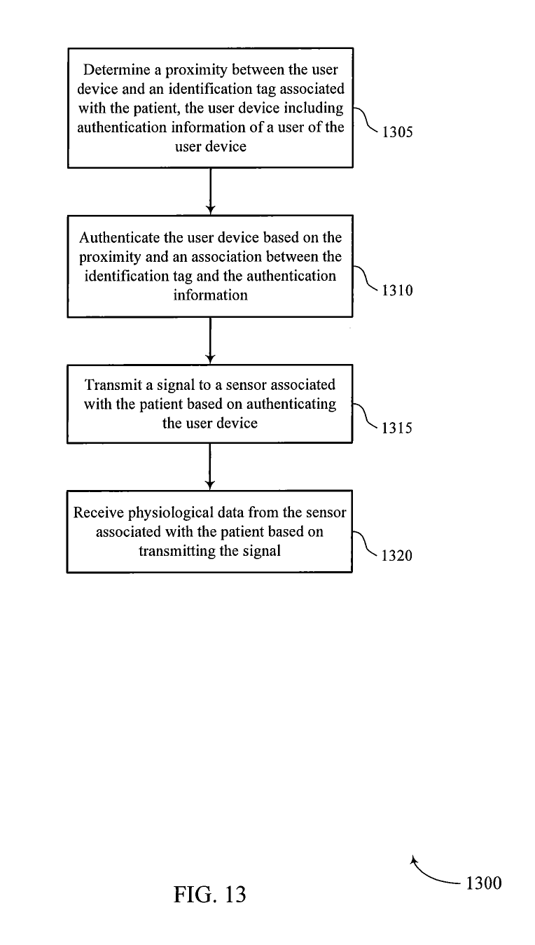

FIG. 13 shows a flowchart illustrating a method 1300 for proximity based data acquisition and display in accordance with aspects of the present disclosure. The operations of method 1300 may be implemented by a user device 115-b or its components as described herein. For example, the operations of method 1300 may be performed by a user device physiological data component as described with reference to FIGS. 5 through 8. In some examples, a user device may execute a set of codes to control the functional elements of the device to perform the functions described below. Additionally or alternatively, the user device may perform aspects of the functions described below using special-purpose hardware.

At 1305 the user device may determine a proximity between the user device and an identification tag associated with the patient, the user device comprising authentication information of a user of the user device. The operations of 1305 may be performed according to the methods described herein. In certain examples, aspects of the operations of 1305 may be performed by a determination component as described with reference to FIGS. 5 through 8.

At 1310 the user device may authenticate the user device based at least in part on the proximity and an association between the identification tag and the authentication information. The operations of 1310 may be performed according to the methods described herein. In certain examples, aspects of the operations of 1310 may be performed by a authentication component as described with reference to FIGS. 5 through 8.

At 1315 the user device may transmit a signal to a sensor associated with the patient based at least in part on authenticating the user device. The operations of 1315 may be performed according to the methods described herein. In certain examples, aspects of the operations of 1315 may be performed by a transmission component as described with reference to FIGS. 5 through 8.

At 1320 the user device may receive physiological data from the sensor associated with the patient based at least in part on transmitting the signal. The operations of 1320 may be performed according to the methods described herein. In certain examples, aspects of the operations of 1320 may be performed by a reception component as described with reference to FIGS. 5 through 8.

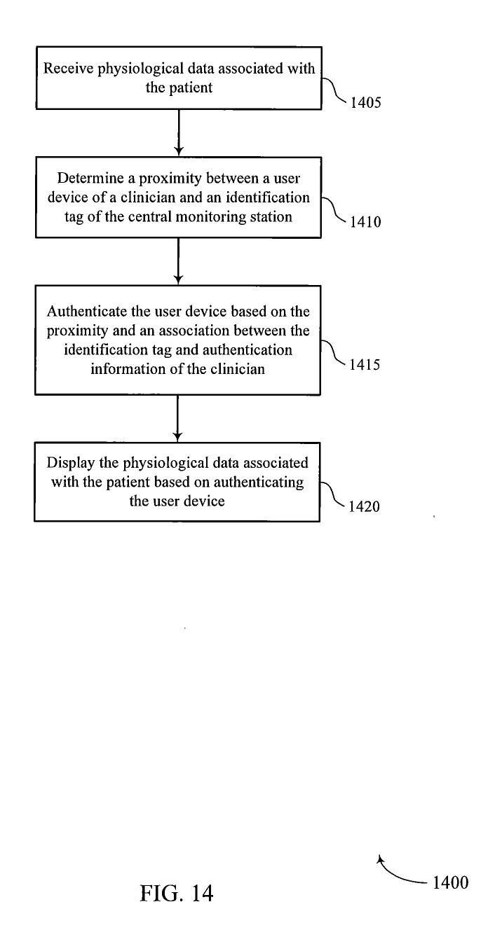

FIG. 14 shows a flowchart illustrating a method 1400 for proximity based data acquisition and display in accordance with aspects of the present disclosure. The operations of method 1400 may be implemented by a central monitoring station 135-a or its components as described herein. For example, the operations of method 1400 may be performed by a CMS physiological data component as described with reference to FIGS. 9 through 12. In some examples, a central monitoring station may execute a set of codes to control the functional elements of the device to perform the functions described below. Additionally or alternatively, the central monitoring station may perform aspects of the functions described below using special-purpose hardware.

At 1405 the central monitoring station may receive physiological data associated with the patient. The operations of 1405 may be performed according to the methods described herein. In certain examples, aspects of the operations of 1405 may be performed by a reception component as described with reference to FIGS. 9 through 12.

At 1410 the central monitoring station may determine a proximity between a user device of a clinician and an identification tag of the central monitoring station. The operations of 1410 may be performed according to the methods described herein. In certain examples, aspects of the operations of 1410 may be performed by a determination component as described with reference to FIGS. 9 through 12.

At 1415 the central monitoring station may authenticate the user device based at least in part on the proximity and an association between the identification tag and authentication information of the clinician. The operations of 1415 may be performed according to the methods described herein. In certain examples, aspects of the operations of 1415 may be performed by a authentication component as described with reference to FIGS. 9 through 12.

At 1420 the central monitoring station may display the physiological data associated with the patient based at least in part on authenticating the user device. The operations of 1420 may be performed according to the methods described herein. In certain examples, aspects of the operations of 1420 may be performed by a display component as described with reference to FIGS. 9 through 12.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an ASIC, an field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration). A processor may in some cases be in electronic communication with a memory, where the memory stores instructions that are executable by the processor. Thus, the functions described herein may be performed by one or more other processing units (or cores), on at least one integrated circuit (IC). In various examples, different types of ICs may be used (e.g., Structured/Platform ASICs, an FPGA, or another semi-custom IC), which may be programmed in any manner known in the art. The functions of each unit may also be implemented, in whole or in part, with instructions embodied in a memory, formatted to be executed by one or more general or application-specific processors.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, digital subscriber line (DSL), or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for receiving physiological data associated with a patient at a user device, comprising:
determining a proximity between the user device and an identification tag associated with the patient, the user device comprising authentication information of a user of the user device;
authenticating the user device based at least in part on the proximity and an association between the identification tag and the authentication information;
transmitting a signal to a sensor associated with the patient based at least in part on authenticating the user device; and
receiving physiological data from the sensor associated with the patient based at least in part on transmitting the signal.

2. The method of claim 1, further comprising:
receiving a subset of physiological data from the sensor associated with the patient based at least in part on a user type of the user of the user device.

3. The method of claim 2, wherein the user type comprises a doctor, a nurse, a physician assistant, or a technician.

4. The method of claim 1, further comprising:
de-authenticating the user device after receiving the physiological data based at least in part on the proximity between the user device and the identification tag exceeding a threshold.

5. The method of claim 1, further comprising:
authenticating the user device is based at least in part on an angle of arrival of the user device.

6. The method of claim 1, wherein the identification tag comprises a radio-frequency identification (RFID) tag.

7. A method for receiving physiological data associated with a patient at a central monitoring station, comprising:
receiving physiological data associated with the patient;
determining a proximity between a user device of a clinician and an identification tag of the central monitoring station;
authenticating the user device based at least in part on the proximity and an association between the identification tag and authentication information of the clinician; and
displaying the physiological data associated with the patient based at least in part on authenticating the user device.

8. The method of claim 7, further comprising:
granting access to a user profile associated with the clinician based at least in part on authenticating the user device, wherein the physiological data associated with the patient is displayed within the user profile.

9. The method of claim 8, further comprising:
revoking access to the user profile associated with the clinician based at least in part on the proximity exceeding a threshold.

10. The method of claim 8, further comprising:
displaying physiological data associated with a plurality of patients within the user profile, wherein the physiological data associated with the plurality of patients is displayed based at least in part on authenticating the user device.

11. The method of claim 7, further comprising:
displaying a subset of the physiological data associated with the patient based at least in part on a credential of the clinician.

12. The method of claim 7, wherein the physiological data associated with the patient is received based at least in part on the proximity between the user device and the identification tag of the central monitoring station.

13. The method of claim 7, further comprising:
authenticating the user device is based at least in part on a media access control (MAC) address associated with the user device.

14. The method of claim 7, further comprising:
transmitting a broadcast message to one or more sensors that indicates a configuration update for the one or more sensors.

15. An apparatus for receiving physiological data associated with a patient at a user device, comprising:
a processor;
memory in electronic communication with the processor; and
instructions stored in the memory and executable by the processor to cause the apparatus to:
determine a proximity between the user device and an identification tag associated with the patient, the user device comprising authentication information of a user of the user device;
authenticate the user device based at least in part on the proximity and an association between the identification tag and the authentication information;
transmit a signal to a sensor associated with the patient based at least in part on authenticating the user device; and
receive physiological data from the sensor associated with the patient based at least in part on transmitting the signal.

16. The apparatus of claim 15, wherein the instructions are further executable by the processor to cause the apparatus to:
receive a subset of physiological data from the sensor associated with the patient based at least in part on a user type of the user of the user device.

17. The apparatus of claim 16, wherein the user type comprises a doctor, a nurse, a physician assistant, or a technician.

18. The apparatus of claim 15, wherein the instructions are further executable by the processor to cause the apparatus to:
de-authenticate the user device after receiving the physiological data based at least in part on the proximity between the user device and the identification tag exceeding a threshold.

19. The apparatus of claim 15, wherein the instructions are further executable by the processor to cause the apparatus to:
authenticate the user device is based at least in part on an angle of arrival of the user device.

20. The apparatus of claim 15, wherein the identification tag comprises a radio-frequency identification (RFID) tag.
